(19) United States
(12) Patent Application Publication
(10) Pub. No.: US 2009/0184057 A1
(43) Pub. Date: Jul. 23, 2009

(54) SYSTEM AND PROCESS FOR REMOVING RESIDUAL PHARMACEUTICAL AND COSMETIC COMPOUNDS FROM DRINKING WATER

(75) Inventor: Dennis L. Mast, Apex, NC (US)

Correspondence Address: COATS & BENNETT, PLLC, 1400 Crescent Green, Suite 300, Cary, NC, 27518, US

(73) Assignee: Clean Water Scientific, Inc., Raleigh, NC (US)

(21) Appl. No.: 12/331,953

(22) Filed: Dec. 10, 2008

Related U.S. Application Data
(63) Continuation-in-part of application No. 12/026,717, filed on Feb. 6, 2008.
Application No. 12/026,717 is a continuation-in-part of application No. 12/015,868, filed on Jan. 17, 2008.

(51) Int. Cl. C02F 1/78; A61L 2/10; C02F 1/58

(52) U.S. Cl. 210/739; 210/748; 210/98

(57) ABSTRACT

A method of removing contaminant compounds such as residual pharmaceutical or cosmetic compounds found in drinking water. The method entails directing the drinking water into a water treatment device and injecting ozone into the drinking water to form a drinking water-ozone mixture. Thereafter the water-ozone mixture is held within a holding tank where the ozone oxidizes various contaminant compounds and reduces their concentration in the drinking water. From the holding tank, the water-ozone mixture is directed to an ultraviolet light chamber where the water is treated with ultraviolet light.

### CROSS REFERENCE TO RELATED APPLICATION

This is a continuation-in-part of U.S. patent application Ser. No. 12/026,717 filed Feb. 6, 2008, and a continuation-in-part of U.S. patent application Ser. No. 12/015,868 filed Jan. 17, 2008. The disclosures of these patent applications are incorporated herein by reference.

### BACKGROUND OF THE INVENTION

It may come as a surprise to many people that drinking water in the United States and other industrialized countries as well, contains minute concentrations of compounds that originate with pharmaceutical products and in some cases cosmetic products. This is especially true in many municipal water supplies. Though many U.S. water basins are contaminated with residues of pharmaceutical and over-the-counter drugs, there apparently is no national strategy to deal with them, nor are there are any effective mandates to test, treat, limit or even advise the general public.

### SUMMARY OF THE INVENTION

The present invention relates to a method of treating drinking water. The method entails mixing ozone with drinking water to form a drinking water-ozone mixture and effectively contacting the contaminants of interest with the ozone for a selected period of time. Thereafter, the method entails treating the water with ultraviolet light.

In one particular embodiment of the present invention, a method is disclosed for removing one or more residual pharmaceutical compounds from drinking water. This method entails directing drinking water having residual pharmaceutical compounds into a water treatment device that can be installed at or within a home, office or other residential or commercial facilities. The drinking water is directed into the water treatment device and the flow of the drinking water is sensed. Ozone is generated by the water treatment device or system. The method entails reducing the concentrations of residual pharmaceutical compounds in the drinking water by mixing ozone with the drinking water. More particularly, in response to there being a flow of drinking water through the water treatment device, ozone is injected into the drinking water to form a drinking water-ozone mixture. Thereafter, the drinking water-ozone mixture is directed into a pressure vessel where the drinking water-ozone mixture is subjected to pressure. In the pressure vessel, the drinking water-ozone mixture is directed up a riser extending within the pressure vessel. At the top of the riser the drinking water-ozone mixture is expelled into a head space defined between a portion of the housing of the pressure vessel and the upper end portion of the riser. Thereafter, the drinking water-ozone mixture moves downwardly through the pressure vessel to an outlet. From the pressure vessel, the drinking water-ozone mixture is directed to a holding tank where the drinking water-ozone mixture is held a selected period of time, enabling the ozone to oxidize the contaminants of interest and effectively reduce the concentration of the contaminants. Thereafter, the drinking water-ozone mixture is directed from the holding tank to a UV light chamber where the drinking water-ozone mixture is treated with ultraviolet light.

In another exemplary process, ozone is mixed with drinking water to reduce the concentrations of such contaminants as Codeine, Acetaminophen, Sulfamethoxazole, Albuteral, Naproxen, and Warfarin. By mixing ozone with the drinking water and contacting these contaminants with ozone, the concentration of any of these contaminants in the drinking water is reduced by approximately 75% or more. Thereafter, the drinking water-ozone mixture is treated with ultraviolet light.

Other objects and advantages of the present invention will become apparent and obvious from a study of the following description and the accompanying drawings which are merely illustrative of such invention.

### BRIEF DESCRIPTION OF THE DRAWINGS

FIG. 1 is a schematic illustration of a water treatment device or system that is used to treat drinking water and to reduce the concentration of contaminants in the drinking water.

FIG. 2 is a perspective view of a pressure vessel utilized in the present process to mix water and ozone.

FIG. 3 is a sectional view of the pressure vessel.

### DETAILED DESCRIPTION

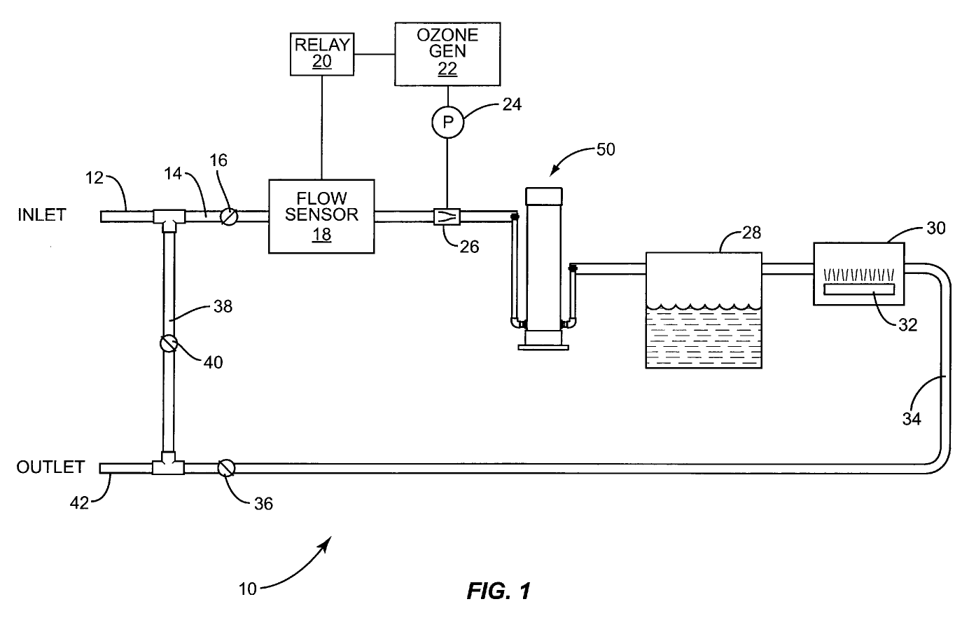

With further reference to FIG. 1, the drinking water treatment unit is shown therein and indicated generally by the numeral 10. Before discussing the components of the water treatment unit 10 in detail, it should be pointed out that the unit can be housed in a cabinet and sized such that it can be conveniently located or positioned in a residential structure, or could be utilized in other buildings and office buildings to treat incoming drinking water from a municipal supply, for example. The water treatment unit 10 is suited for both residential and commercial use.

Referring to FIG. 1 specifically, the water treatment unit 10 includes an inlet 12 that is communicatively connected to a conduit 14. Generally, in one embodiment, the inlet 12 is associated with a cabinet structure that houses the various components of the water treatment unit 10 and the conduit 14 extends at least through portions of the cabinet structure. Disposed in the conduit 14 is an on-off valve 16. Downstream from the on-off valve 16 is a flow sensor 18. The function of the flow sensor 18 is to sense the flow of drinking water through conduit 14 and to actuate or control one or more other components of the treatment unit 10 that is located downstream from the flow sensor 18.

Water treatment unit 10 includes an ozone generator 22 for generating ozone that is injected and mixed into the drinking water passing in conduit 14. In some embodiments a pump 24 can be utilized to pump or move ozone from the ozone generator 22 to a point where the ozone is mixed with the drinking water passing conduit 14. In the case of one embodiment, a venturi 28 is disposed in the conduit 14 and an ozone delivery line is operatively interconnected between the ozone generator and the venturi 28 for directing ozone into the conduit 14.

The water treatment unit 10 in one embodiment is designed to periodically or intermittently inject ozone into the drinking water passing in conduit 14. In the embodiment illustrated, ozone is injected into the drinking water passing conduit 14 in response to the flow sensor 18 sensing flow of drinking water in the conduit. That is, as illustrated in FIG. 1, the flow sensor 18 senses the flow of drinking water passing in conduit 14 and then actuates a relay 20 that in turn actuates the ozone generator 22 causing ozone to be generated and injected into the drinking water. It is appreciated by those skilled in the art, that various control devices can be utilized to actuate and control the ozone generator 22 in response to the flow sensor 18 sensing flow. In one embodiment, the ozone generator 22 is adjusted or calibrated to output a flow of ozone in response to the flow sensor 18 sensing flow. In other embodiments, the control system can be more sophisticated where the ozone generated and injected into the conduit 14 is a function of the volumetric flow rate of drinking water passing through the flow sensor 18. It is postulated that a precise correlation between the output of ozone from the generator 22 and the flow rate of drinking water passing through the flow sensor is not essential or required. In one embodiment, the purpose of the flow sensor and its actuation of the ozone generator 22 is to generally assure that ozone is not being wasted or injected into the system when there is no significant flow of drinking water passing through the water treatment unit 10. In order for the process to be effective in removing targeted contaminants, it is postulated that there need not be a precise injection of ozone into the drinking water based on drinking water flow. It is appreciated, that for the process to be effective, that ozone is mixed with the drinking water in adequate amounts to reduce the concentration of targeted contaminants.

As the ozone is injected via the venturi 28 into the drinking water, the venturi 28 functions to at least partially mix the ozone with the drinking water. This forms a drinking water-ozone mixture in the conduit 14.

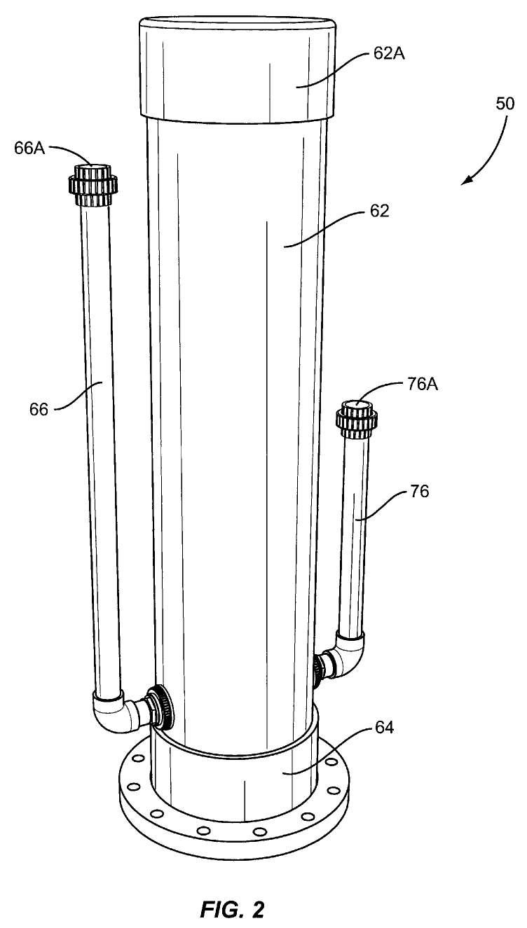

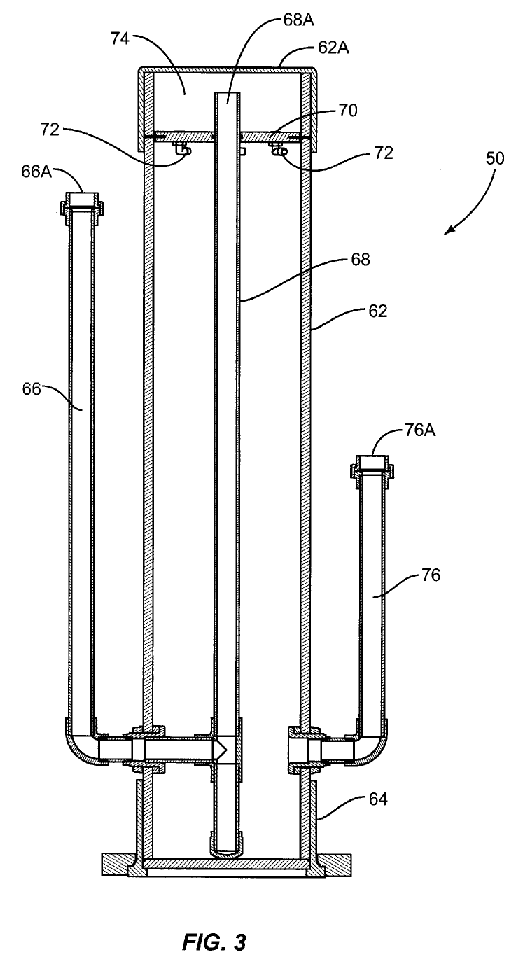

Located downstream of the venturi 28 is a pressure vessel 50 which also functions to mix the ozone and drinking water. Pressure vessel 50 maintains the drinking water-ozone mixture under pressure as the mixture passes through the pressure vessel. Pressure vessel 50 is shown in more detail in FIGS. 2 and 3 and will be discussed subsequently herein.

Located downstream from the pressure vessel 50 is a holding tank 28 that holds the drinking water-ozone mixture for a selected period of time. This enables the ozone to contact the contaminants and to generally oxidize the contaminants and reduce their concentrations. The residency time in the holding tank 28 can vary. In one embodiment, the residency time is approximately 10 minutes to approximately 15 minutes. The size and volume of the holding tank can vary, but when the water treatment unit 10 is used in a residential application, it is contemplated that a holding tank of approximately 15 gallons is adequate to provide an effective residency time for the drinking water-ozone mixture under ordinary conditions.

Disposed downstream of the holding tank 28 is a UV light chamber 30. Mounted in the UV light chamber 30 are one or more UV lights 32. As the water or the drinking water-ozone mixture passes through the UV light chamber 30, the UV lights 32 treat the drinking water and effectively nullify any substantial concentration of ozone in the drinking water. The use of UV light performs a polishing function. In most cases there is no substantial amount of ozone in the water once the water reaches the UV chamber 30. This is because the half life of ozone is approximately 10 minutes and the drinking water-ozone mixture is usually held 10 minutes or more.

After leaving the UV light chamber 30, the drinking water continues to pass through conduit 34 and through an on/off valve 36. Conduit 34 extends beyond on/off valve 36 and an outlet 42 is provided for directing the treated drinking water from the water treatment unit 10. As shown in FIG. 1, a bypass conduit 38 is provided downstream of the inlet 12 and upstream of the outlet 42. An on/off valve 40 is placed in the bypass conduit 38.

Now turning to a discussion of the pressure vessel 50 shown in FIGS. 2 and 3, the pressure vessel includes a housing 62 supported on a base 64. Housing 62 includes an upper cap 62A that is secured about the upper terminal end of the housing. Pressure vessel 50 includes an inlet tube 66. As seen in FIGS. 2 and 3, inlet tube 66 extends alongside the housing 62 and includes an upper terminal end 66A that is substantially elevated with respect to the base 64 of the pressure vessel 50. Internally within the housing 62 is a riser 68. Riser 68 is communicatively connected to the inlet tube 66 and includes an upper terminal end 68A. Disposed below outlet 68A is a separation plate 70 that extends around the riser 68 and engages the interior wall of the housing 62. A head space 74 is defined between the cap 62A and the plate 70. Plate 70 includes a series of circumferentially spaced openings (not shown) formed therein that permit the drinking water-ozone mixture to move from the head space 74 downwardly pass the plate 70 into the lower portions of the pressure vessel 50. Connected to each opening in the plate 70 about the underside thereof is a disburser 72. The dispersers 72 are generally L-shaped dispersing elements that turn the drinking water-ozone mixture in a direction generally horizontally and toward the wall of the housing 62 such that the drinking water-ozone mixture is disbursed generally tangentially to the inner wall of the housing 62. The number of openings in plate 70 and the number of dispersers 72 can vary. Generally however they are uniformly spaced around the axis of the riser 68.

The pressure vessel 50 also includes an outlet tube 76. Outlet tube 76 is communicatively connected to the interior of the pressure vessel 50 about a lower portion. See FIG. 3. Outlet tube 76 includes an outlet 76A.

In the present process, the drinking water-ozone mixture enters the pressure vessel 50 via inlet tube 66. Once in the inlet tube 66, the drinking water-ozone mixture travels downwardly and into the lower portion of the riser 68. Then the drinking water-ozone mixture is pumped or moved upwardly through riser 68 to where the mixture is disbursed from the upper outlet 68A. The mixture is disbursed under pressure and impinges or impacts against the inner surface of cap 62. Thereafter the mixture falls downwardly into the head space 74 and is disbursed from the head space through openings in the plate 70 and through disperses 72. Because of the orientation of disperses 72, the drinking water-ozone mixture generally follows a swirling pattern as it moves downwardly around the riser 68. The drinking water-ozone mixture accumulates about the bottom of the pressure vessel 50 and is effectively discharged therefrom through the outlet tube 76.

It should be appreciated that there are various designs that can be incorporated into the upper portion of the pressure vessel 50 to cause the drinking water-ozone mixture to swirl downwardly through the pressure vessel. For example in one embodiment, the pressure vessel may include baffles that impart a swirling motion to the mixture as it moves from the upper portion of the pressure vessel to a lower portion.

The pressure maintained in the head space 74 can vary. In one embodiment, the pressure in the head space 74 ranges from approximately 35 psi to approximately 65 psi.

The system and process for removing residual pharmaceutical compounds and other residual compounds such as residual cosmetic product compounds is effective in removing certain of these residual compounds. For example, in one test the results indicate a sufficient reduction in a series of residual compounds including Codeine, Acetaminophen, Sulfamethoxazole, Albuteral, Naproxen, and Warfarin. In these tests the drinking water flow was approximately 2.4 gallons/min. The inlet pressure was approximately 55 psi and the outlet pressure was approximately 15 to 20 psi. In order to provide a substantial dose of ozone at any flow rate, the pump 24 was utilized to deliver ozone at a pressure of approximately 70 psi which is greater than the system operating pressure. The high pressure ozone pump 24 is capable of delivering a substantial amount of ozone produced by the generator 22 at any system flow rate. The drinking water-ozone mixture was directed into the holding tank 28 and the mixture was held therein for approximately 15 minutes. This enabled the ozone to have sufficient contact time with the contaminants to oxidize and effectively reduce the concentration of certain contaminants. After contact in the holding tank 28, the drinking water or the drinking water-ozone mixture was directed to the UV light chamber 30 for treatment by the UV lights 32. In the case of the six contaminants listed above, there was a substantial reduction in their concentrations. For example, the untreated amount of Codeine in the drinking water was 0.65 ppb. There was no detectable Codeine found in the treated drinking water which resulted in a 100% reduction in the concentration of Codeine. For Acetaminophen, the untreated concentration was 0.77 ppb and the treated concentration was 0.06 ppb, yielding a 93% reduction in the concentration. In the case of Sulfamethoxazole, the untreated concentration was 0.49 ppb and there was no detectable amount in the treated drinking water. This yielded a 100% reduction in concentration. For Albuteral, the untreated concentration was 0.14 ppb and there was no detectable amount of Albuteral in the treated drinking water. This resulted in a 100% reduction in concentration. The same holds true for Naproxen. Here the initial concentration was 0.88 ppb with no detectable amounts in the treated drinking water, yielding a 100% reduction in concentration. Finally in the case of Warfarin, the initial or untreated concentration was 0.86 ppb and the concentration in the treated drinking water was 0.02, yielding a 98% reduction in concentration.

From the foregoing, it is appreciated that the present system and process is useful in treating municipal water supplies and other water supplies that include residual compound from pharmaceutical products, cosmetic products, and other such products. The entire system can be housed within a cabinet and installed within a dwelling or other residential or commercial structure.

The present invention may, of course, be carried out in other ways than those specifically set forth herein without departing from essential characteristics of the invention. The present embodiments are to be considered in all respects as illustrative and not restrictive, and all changes coming within the meaning and equivalency range of the appended claims are intended to be embraced therein.

1. A method of removing one or more residual pharmaceutical compounds from drinking water comprising:
a. directing drinking water having residual pharmaceutical compounds into an apparatus for treating the drinking water;
b. sensing the flow of drinking water having the residual pharmaceutical compounds passing through the apparatus;
c. generating ozone with an ozone generator;
d. reducing the concentrations of residual pharmaceutical compounds in the drinking water by mixing ozone with the drinking water, reducing the concentration of the residual pharmaceutical compounds comprising:
i. injecting ozone into the drinking water to form a drinking water-ozone mixture wherein the injection of ozone into the drinking water is in response to sensing drinking water passing through the apparatus;
ii. directing the drinking water-ozone mixture into a pressure vessel and into a riser extending upwardly through the pressure vessel;
iii. discharging the drinking water-ozone mixture into a head space above a terminal end of the riser;
iv. directing the drinking water-ozone mixture from the head space downwardly through the pressure vessel;
v. discharging under pressure the drinking water-ozone mixture from the pressure vessel;
vi. directing the drinking water-ozone mixture from the pressure vessel to a holding tank;
vii. holding the drinking water-ozone mixture in a holding tank for a selected period of time;
e. directing the drinking water-ozone mixture from the holding tank to a UV light treatment chamber;
f. treating the drinking water-ozone mixture with ultraviolet light and producing treated drinking water; and
g. directing the treated drinking water from the apparatus.

2. The method of claim 1 wherein the drinking water includes one or more of the residual pharmaceutical compounds taken from the group consisting of Codeine, Acetaminophen, Sulfamethoxazole, Albuteral, Naproxen, and Warfarin; wherein each residual pharmaceutical compound in the drinking water includes an initial concentration; and wherein the method reduces the concentration of the one or more residual pharmaceutical compounds by 75% or more.

3. The method of claim 1 including holding the drinking water-ozone mixture in the holding tank for a period of approximately ten to approximately fifteen minutes.

4. The method of claim 1 wherein injecting ozone into the drinking water includes injecting ozone into a conduit having a venturi for facilitating the mixing of the ozone with the drinking water.

5. The method of claim 1 wherein directing the drinking water-ozone mixture from the head space downwardly through the pressure vessel includes directing the drinking water-ozone mixture along a swirling flow path towards an outlet in the pressure vessel.

6. The method of claim 1 wherein there is provided an elongated inlet tube disposed adjacent the pressure vessel and wherein the method includes directing the drinking water-ozone mixture into an elevated inlet end of the inlet tube.

7. The method of claim 1 including maintaining the pressure in the head space at approximately 35 psi to approximately 65 psi.

8. A method of removing Codeine, Acetaminophen, Sulfamethoxazole, Albuteral, Naproxen, and Warfarin from drinking water comprising:
a. directing drinking water into a water treatment device wherein the drinking water includes an initial concentration of Codeine, Acetaminophen, Sulfamethoxazole, Albuteral, Naproxen, or Warfarin;
b. injecting ozone into the drinking water and mixing the ozone with the drinking water to form a drinking water-ozone mixture;
c. holding the drinking water-ozone mixture in a holding container for a time period that is sufficient to reduce the initial concentration of Codeine, Acetaminophen, Sulfamethoxazole, Albuteral, Naproxen, or Warfarin by 75% or more;
d. after mixing ozone with the drinking water, directing the drinking water-ozone mixture to a UV light treatment chamber; and
e. treating the drinking water-ozone mixture with UV light.

9. The method of claim 8 including sensing the flow of drinking water flowing through the water treatment device and in response to sensed drinking water flow, injecting ozone into the drinking water to form the drinking water-ozone mixture.

10. The method of claim 8 wherein after injecting the ozone into the drinking water to form the drinking water-ozone mixture, directing the drinking water-ozone mixture to a pressure vessel and into a riser that extends upwardly through the pressure vessel; and discharging the drinking water-ozone mixture from the terminal end of the riser into a head space formed in the pressure vessel; and thereafter allowing the drinking water-ozone mixture to move down through the pressure vessel and out an outlet thereof.

11. The method of claim 10 including swirling the drinking water-ozone mixture around the riser as the drinking water-ozone mixture moves downwardly from the head space within the pressure vessel to the outlet of the pressure vessel.

12. The method of claim 11 including directing the drinking water through a conduit and sensing the flow of drinking water through the conduit; in response to flow being sensed in the conduit, injecting ozone into the flowing drinking water at a point downstream from where the flow is sensed.

13. The method of claim 8 including pumping ozone under pressure into a conduit and mixing the ozone with the drinking water to form the drinking water-ozone mixture.

14. A method of removing one or more residual compounds from drinking water where the residual compounds result from pharmaceutical or cosmetic products, the method comprising:
a. directing drinking water into a water treatment device where the drinking water includes the residual compounds resulting from pharmaceutical or cosmetic products;
b. directing the drinking water through a conduit in the water treatment device;
c. sensing the flow of drinking water flowing through the conduit;
d. injecting ozone into the flowing drinking water in response to the flow of drinking water in the conduit being sensed;
e. mixing the ozone with the drinking water to form a drinking water-ozone mixture;
f. holding the drinking water-ozone mixture for a time sufficient to reduce the concentration of the one or more residual compounds by 75% or more;
g. directing the drinking water-ozone mixture to a UV light chamber; and
h. treating the drinking water-ozone mixture with ultraviolet light.

15. The method of claim 14 including directing the drinking water-ozone mixture into a pressure vessel and through a conduit in the pressure vessel and from the conduit in the pressure vessel into a space defined between the terminal end of the conduit in the pressure vessel and an internal wall of a housing in part of the pressure vessel.

16. The method of claim 14 including directing the drinking water-ozone mixture into a pressure vessel and pressurizing the drinking water-ozone mixture in the pressure vessel.

17. The method of claim 16 including directing the drinking water-ozone mixture up through a riser disposed internally within the pressure vessel and out a terminal end of the riser where the drinking water-ozone mixture is forced against a wall of a housing that forms a part of the pressure vessel, and then directing the drinking water-ozone mixture downwardly through the pressure vessel to an outlet disposed in a lower portion of the pressure vessel.

18. The method of claim 17 including swirling the drinking water-ozone mixture as the drinking water-ozone mixture moves downwardly through the pressure vessel.

19. The method of claim 14 wherein the drinking water-ozone mixture includes one or more residual pharmaceutical compounds taken from the groups consisting of Codeine, Acetaminophen, Sulfamethoxazole, Albuteral, Naproxen, and Warfarin; and wherein the method includes reducing the concentration of each of the residual pharmaceutical compound to approximately 75% or more.

20. A water treatment apparatus for treating drinking water comprising:
a. a drinking water inlet;
b. a conduit for directing the drinking water through the water treatment apparatus;
c. a flow sensor in the water treatment apparatus operative to sense the flow of water through the conduit;
d. an ozone generator for generating ozone;
e. an ozone injection line operatively connected between the ozone generator and the conduit for directing ozone into the conduit;
f. the generator being actuated by the flow sensor such that the generator generates ozone in response to the flow sensors sensing flow of water through the conduit;
g. a pressure vessel located downstream from where the ozone is mixed with the drinking water for receiving a drinking water-ozone mixture;
h. wherein the pressure vessel includes a chamber and an elongated riser extending upwardly through the chamber and wherein there is a head space defined in the upper portion of the chamber for receiving the drinking water-ozone mixture discharged from the riser, and wherein the pressure vessel includes an outlet for directing the drinking water-ozone mixture from the pressure vessel; and
i. a UV light treatment chamber located downstream from the pressure vessel.

21. The water treatment apparatus of claim 20 wherein the pressure vessel includes a top and a plate spaced below the top and extending through the pressure vessel such that the head space is defined between the top and the plate; and wherein the plate includes a series of dispersers for dispersing the drinking water-ozone mixture from the head space into a portion of the pressure vessel disposed below the plate.
